(19) United States
(12) Patent Application Publication
(10) Pub. No.: US 2008/0287982 A1
(43) Pub. Date: Nov. 20, 2008

(54) Catheters for electrolytically detachable embolic devices

(75) Inventor: John H. Harreld, Guerneville, CA (US)

Correspondence Address: ROBINS & PASTERNAK, 1731 EMBARCADERO ROAD, SUITE 230, PALO ALTO, CA, 94303, US

(73) Assignee: Boston Scientific Scimed, Inc.

(21) Appl. No.: 12/151,501

(22) Filed: May 7, 2008

Related U.S. Application Data
(60) Provisional application No. 60/930,436, filed on May 16, 2007.

(51) Int. Cl. A61M 25/00

(52) U.S. Cl. 606/191

(57) ABSTRACT

Disclosed herein are catheters for electrolytically detachable vaso-occlusive devices. Also disclosed are vaso-occlusive assemblies comprising these catheters and methods of using these catheters and assemblies.

### CROSS-REFERENCE TO RELATED APPLICATIONS

This application claims the benefit of U.S. provisional patent application No. 60/930,436, filed May 16, 2007, the disclosure of which is incorporated by reference in its entirety for all purposes.

### TECHNICAL FIELD

Catheters for use in repairing aneurysms are described. In particular, catheter designs that enhance electrolytic detachment of vaso-occlusive devices are described.

### BACKGROUND

An aneurysm is a dilation of a blood vessel that poses a risk to health from the potential for rupture, clotting, or dissecting. Rupture of an aneurysm in the brain causes stroke, and rupture of an aneurysm in the abdomen causes shock. Cerebral aneurysms are usually detected in patients as the result of a seizure or hemorrhage and can result in significant morbidity or mortality.

Catheters are routinely used to deliver implantable embolic devices, such as vaso-occlusive coils, to cerebral and other aneurysms. Flexible, single and multi-layered catheters suitable for delivering embolic devices to the brain have been described. See, e.g., U.S. Pats. No. 4,739,768; 5,454,795; 5,702,373; 6,030,369; and 6,824,553. The catheters may be guided to the site through the use of guidewires (see U.S. Pat. No. 4,884,579) or by flow-directed means such as balloons placed at the distal end of the catheter. For example, a vascular access introducer sheath may be threaded over the guidewire and into the vessel. The wire is removed and the introducer sheath is left in place as a delivery device to provide co-axial access.

Once the site has been reached, the catheter lumen is cleared by removing the guidewire (if a guidewire has been used), and one or more coils are placed into the proximal open end of the catheter and advanced through the catheter with a pusher. When the coil reaches the distal end of the catheter, it is discharged from the catheter by the pusher into the vascular site, for example by electrolytic detachment mechanisms (see, U.S. Pat. Nos. 5,122,136; 5,354,295; 6,620,152; 6,425,893; and 5,976,131, all to Guglielmi et al., describing electrolytically detachable embolic devices; U.S. Pat. No. 6,623,493 describing vaso-occlusive member assembly with multiple detaching points).

In such electrolytically detachable devices, the coil is bonded via a metal-to-metal joint to the distal end of the pusher. The pusher and coil are made of dissimilar metals. The coil-carrying pusher is advanced through the catheter to the site and a small electrical current is passed through the pusher-coil assembly. See, e.g., U.S. Pat. No. 6,059,779. The current causes the joint between the pusher and the coil to be severed via electrolysis. The pusher may then be retracted leaving the detached coil within the vessel. However, detachment times for electrolytic detachable devices can be on the order of 20-30 seconds or even longer when the detachment junction is not sufficiently distal to (extruded from) the delivery catheter.

Thus, there is a need for catheter designs that provide rapid deployment of electrolytically detachable vaso-occlusive devices, even when the detachment junction is within the delivery mechanism.

### SUMMARY

The present disclosure relates to catheter designs that allow for rapid electrolytic detachment of implantable devices, even when the electrolytic detachment junction (detachment zone) is within the catheter. Assemblies comprising these catheters, methods of using and making these devices are also described.

In one aspect, disclosed herein is a catheter for use in delivering electrolytically detachable vaso-occlusive devices, the catheter having a distal region and a lumen therethrough, wherein, in the distal region, the sidewalls of the catheter comprise one or more apertures therein. The apertures may comprise one or more slots or holes, for example, longitudinal slots and/or axially aligned or staggered holes.

In any of the catheters described herein, the distal region of the catheter may comprise a porous membrane.

In another aspect, described herein is a catheter, wherein the lumen of the catheter comprises an electrolyte solution, for example, saline or phosphate buffered saline. In certain embodiments, catheters having a lumen comprising electrolytes therein further comprises one or more apertures in the distal region of the sidewalls and/or a porous membrane, for example at the distal region.

In another aspect, a catheter for use in delivering electrolytically detachable vaso-occlusive devices, the catheter having a distal region, a lumen therethrough and means for maintaining current flow in the distal region of the catheter to obtain minimal resistive drop between the detachment site and the subject's blood. In certain embodiments, the current flow is maintained at approximately 1 mA.

In yet another aspect, described herein is a vaso-occlusive assembly comprising a vaso-occlusive device comprising an electrolytically detachable junction; and any of the catheters as described herein.

In yet another aspect, described herein is a method of at least partially occluding an aneurysm, the method comprising the step of introducing a vaso-occlusive device into the aneurysm using any of the vaso-occlusive assemblies described herein.

These and other embodiments will readily occur to those of skill in the art in light of the disclosure herein.

### BRIEF DESCRIPTION OF THE FIGURES

FIGS. 1A, 1B and 1C are overviews of exemplary catheter modifications that enhance electrolytic detachment. FIG. 1A shows the distal region of a catheter with longitudinal slots in the catheter sidewall. FIG. 1B shows the embodiment of FIG. 1A with a porous membrane layer covering the longitudinal slots. The porous membrane layer may be flush with the distal end of the catheter, may extend beyond the distal end or may terminate prior the distal end of the catheter. FIG. 1C shows an exemplary embodiment in which the distal region of the catheter comprises a porous membrane layer.

FIGS. 2A and 2B are overviews of additional exemplary catheter modifications as described herein. FIG. 2A shows the distal region of a catheter with axially aligned holes in the sidewalls. FIG. 2B shows a staggered hole design. An optional porous membrane layer (not shown) may also be included.

FIG. 3 is a side-view, cross-section showing an exemplary assembly for delivering an electrolytically detachable vaso-occlusive coil using a catheter as described herein.

### DETAILED DESCRIPTION

Catheter designs for delivery of vaso-occlusive devices comprising electrolytic detachment junctions and assemblies comprising these catheter designs are described. The catheters described herein can be utilized in vascular and neurovascular indications and are particularly useful in delivering embolic devices to aneurysms located within, for example, small-diameter, curved or otherwise difficult to access vasculature, such as cerebral aneurysms. Methods of making and using these catheters and assemblies comprising these catheters are also described.

Currently, the gold-standard method of delivering vaso-occlusive devices is via electrolytic detachment (e.g., GDC coils). While electrolytic detachment solves the drawbacks of earlier mechanical detachments (e.g., the need for the mechanism to be fully inside the catheter in order to remain engaged), electrolytic detachment can be inhibited when the detachment junction is not fully extruded from the catheter. In particular, when the detachment zone electrode surface and the blood electrolytes are physically separated by incomplete extrusion of the detachment zone, there is an apparent increase in the resistive voltage drop between the detachment zone and blood. When the total voltage measured by the power supply saturates at the designated limit, the current actually running to the detachment junction is below the intended 1 mA. The lower than intended current in turn leads to slower metal dissolution rates and longer detachment times.

Described herein are catheter designs that lower the resistance pathway for electrolyte ions to the electrolytic detachment junction (e.g., one that is contained at least partially within the catheter). Accordingly, the total voltage measured by the power supply is not saturated or above its limit and the current can be maintained at or near the intended 1 mA levels. The catheters described herein allow for ready and predictable electrolytic detachment of vaso-occlusive devices even in cases when the device is aligned such that the detachment junction is contained within the delivery device (e.g., microcatheter sheath). Thus, the designs described herein reduce undesirably long and variable electrolytic detachment times for embolic devices.

All publications, patents and patent applications cited herein, whether above or below, are hereby incorporated by reference in their entirety.

It must be noted that, as used in this specification and the appended claims, the singular forms “a”, “an”, and “the” include plural referents unless the content clearly dictates otherwise. Thus, for example, reference to a catheter comprising “an aperture” in the sidewall(s) of the distal region of the catheter includes catheters comprising of two or more such sidewall apertures.

As used herein, the term “catheter” refers to any hollow, flexible tube that can be inserted into the vasculature. The term includes any part of a catheter, including the main body and/or surrounding sheath(s) that are commonly used in neurovascular indications.

The catheter designs described herein include catheters (e.g., microcatheter sheaths) having one or more apertures (e.g., channels, pores, holes, slots, etc.) in the sidewalls of the catheter in the distal region which apertures allow for electrolytes to flow into the distal region of the catheter and enhance the current transmitted to the electrolytic detachment zone. The catheters may be single or multi-layered. See, e.g., U.S. Pat. No. 6,030,369.

Any number of channels may be incorporated into the sidewalls of the catheter. Furthermore, the channels may be of any size or shape or combinations of different sizes and shapes. Thus, although depicted in the drawings as longitudinal slots or holes (pores), the catheters described herein may include any combination of slots and/or holes in the same or different sizes and in the same or different orientations. The size of the sidewall apertures can be readily selected, depending on factors such as the location or size of the intended target, the amount and type of blood flow in the intended target and the like. For example, larger diameter catheters may include larger channels.

Also described herein are catheter designs in which the distal end of the lumen of the catheter comprises an ionic solution therein, for example, by flushing the catheter before, during and/or after insertion of the vaso-occlusive device to be delivered. In these designs, the distal end of the catheter may also include any combination of apertures and/or porous membrane(s).

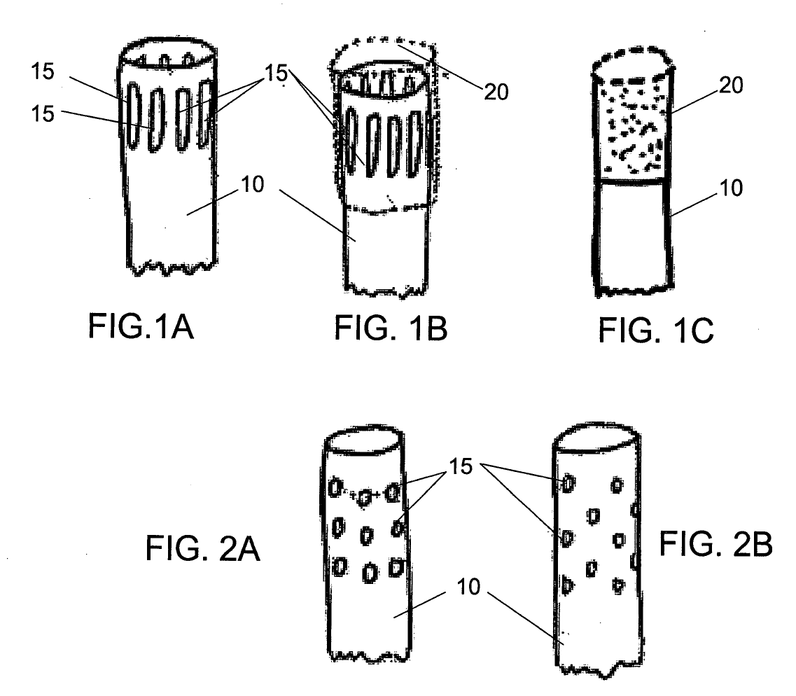

The catheters described herein may be made of any biologically compatible material(s), including for example, polyethylene, polypropylene, Nylon, polyurethane, polyimides, polyvinyl chloride, polysulfones, polyfluorocarbons, polyethylene terephthalate, their mixtures, copolymers; and/or polyester elastomers. In certain embodiments, the distal region of the catheter comprising the sidewall apertures is made of a different material than the remainder of the catheter, for example, when the distal region comprises a porous membrane layer (see, FIGS. 1B and 1C).

FIG. 1A shows the distal region of an exemplary catheter 10 having multiple longitudinal slots 15 in the sidewalls of the catheter. FIG. 1B shows an optional porous membrane 20 covering the slots 15 and extending beyond the distal end of the catheter 10. FIG. 1C shows an embodiment in which the distal region of the catheter 10 is a porous membrane 20.

When present, the porous membrane may be made of a variety of synthetic and/or natural materials, including, but not limited to, nylon, polyurethane, cotton, silk, and combinations thereof. As with the apertures, the size of the pores of the membrane can be selected based on the desired flow into or out of the lumen of the catheter (e.g., electrolytes).

Furthermore, it will be apparent that when used with a catheter design comprising one more channels in the sidewalls of the distal region, the optional additional porous element may cover some, all or none of the channels. For instance, as shown in FIG. 1B, the porous element may extend past the distal end of the catheter. Alternatively, the porous element may be flush with the distal end of the catheter or may be trimmed so that it does not extend to the distal end of the catheter. The proximal end of the porous element may extend proximally to cover all or some of the channels. The skilled artisan can readily determine the positioning of the porous membrane suitable for the selected indication.

FIGS. 2A and B show embodiments in which the channels in the sidewalls of the distal end of the catheter 10 are holes 15. FIG. 2A shows axially aligned holes. FIG. 2B shows staggered hole designs. As noted above, a porous element may cover some or all of the holes.

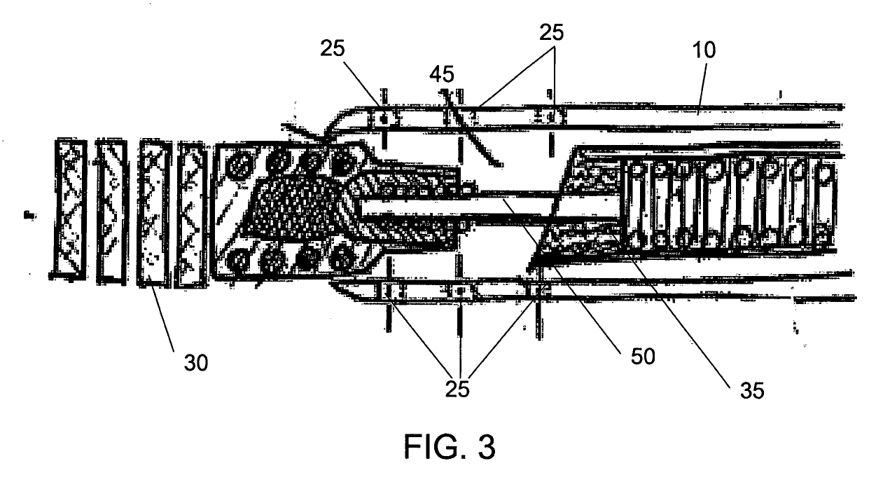

FIG. 3 shows an assembly including a catheter 10 as described herein including multiple channels 25, a vaso-occlusive device 30 attached to a pusher element 35 via an electrolytical detachment junction 50.

Also shown in FIG. 3 is a design in which the lumen of catheter (particularly at the distal end) contains an electrolyte solution that enhances electrolytic detachment, for example in situations where the detachment zone is incompletely deployed from the catheter. Although depicted in FIG. 3 as a design having apertures in the sidewalls of the catheter, it will be apparent that such apertures are optional and that designs containing an electrolyte solution in the lumen of the catheter may have solid sidewalls.

Any electrolyte solution may be used, including, but not limited to, saline and phosphate buffered saline. A skilled artisan can readily determine other solutions exhibiting the same electrolyte properties as saline and phosphate buffered buffered saline.

Furthermore, the electrolyte solution can be placed in the catheter prior to, during and/or after introduction of the vaso-occlusive device. For example, the lumen of the catheter as described herein may be flushed with a selected electrolyte solution (e.g., saline) prior to insertion of the vaso-occlusive device. In addition, as an alternative to or in combination with flushing the catheter prior to insertion of the vaso-occlusive device, the catheter may be flushed when the vaso-occlusive device is within the lumen, e.g., by flushing of the lumen and/or employing catheters comprising one or more fluid conduits in the walls of the catheter.

The catheters described herein can be adapted to be used with any vaso-occlusive devices. In a preferred embodiment, the vaso-occlusive device comprises a helical wire coil having windings that may be dimensioned to engage the walls of the vessels. (See, e.g., U.S. Pat. No. 4,994,069 to Ritchart et al.). Variations of such devices include polymeric coatings or attached polymeric filaments have also been described. See, e.g., U.S. Pat. Nos. 5,226,911; 5,935,145; 6,033,423; 6,280,457; 6,287,318; and 6,299,627. In addition, coil designs including stretch-resistant members that run through the lumen of the helical vaso-occlusive coil have also been described. See, e.g., U.S. Pat. Nos. 5,582,619; 5,833,705; 5,853,418; 6,004,338; 6,013,084; 6,179,857; and 6,193,728.

Suitable vaso-occlusive devices include, but not limited to, metal and/or polymeric devices. Suitable metals and metal alloys include the Platinum Group metals, especially platinum, rhodium, palladium, rhenium, as well as tungsten, gold, silver, tantalum, and alloys of these metals. The core element may also comprise of any of a wide variety of stainless steels. Very desirable materials of construction, from a mechanical point of view, are materials that maintain their shape despite being subjected to high stress including but not limited to “super-elastic alloys” such as nickel/titanium alloys (48-58 atomic % nickel and optionally containing modest amounts of iron); copper/zinc alloys (38-42 weight % zinc); copper/zinc alloys containing 1-10 weight % of beryllium, silicon, tin, aluminum, or gallium; or nickel/aluminum alloys (36-38 atomic % aluminum). Particularly preferred are the alloys described in U.S. Pat. Nos. 3,174,851; 3,351,463; and 3,753,700. Especially preferred is the titanium/nickel alloy known as “nitinol.”

The vaso-occlusive devices may be of any structure, for example, vaso-occlusive devices of tubular structures, for examples, braids, coils, combination braid and coils and the like. Thus, although depicted in the Figures described below as a coil, the vaso-occlusive device may be of a variety of shapes or configuration includes, but not limited to, braids, knits, woven structures, tubes (e.g., perforated or slotted tubes), cables, injection-molded devices and the like. See, e.g., U.S. Pat. No. 6,533,801 and International Patent Publication WO 02/096273. The vaso-occlusive device may change shape upon deployment, for example change from a constrained linear form to a relaxed, three-dimensional (secondary) configuration. See, also, U.S. Pat. No. 6,280,457. In a preferred embodiment, the core element comprises a metal wire wound into a primary helical shape.

The devices are often introduced into a selected site using the procedure outlined below. This procedure may be used in treating a variety of maladies. For instance in the treatment of an aneurysm, the aneurysm itself will be filled (partially or fully) with electrolytically detachable devices.

Conventional catheter insertion and navigational techniques involving guidewires or flow-directed devices may be used to access the site with a catheter as described herein. For use in peripheral or neural surgeries, the catheter will normally be about 100-200 cm in length, more normally 130-180 cm in length. The diameter of the delivery mechanism is usually in the range of 0.25 to about 0.90 mm. Briefly, occlusive devices (and/or additional components) are typically loaded into a carrier for introduction into the delivery catheter and introduced to the chosen site.

A selected site is reached through the vascular system using a collection of specifically chosen catheters and/or guide wires. It is clear that should the site be in a remote site, e.g., in the brain, methods of reaching this site are somewhat limited. One widely accepted procedure is found in U.S. Pat. No. 4,994,069 to Ritchart, et al. It utilizes a fine endovascular catheter such as is found in U.S. Pat. No. 4,739,768, to Engelson. First of all, a large catheter is introduced through an entry site in the vasculature. Typically, this would be through a femoral artery in the groin. Other entry sites sometimes chosen are found in the neck and are in general well known by physicians who practice this type of medicine. Once the introducer is in place, a guiding catheter is then used to provide a safe passageway from the entry site to a region near the site to be treated. For instance, in treating a site in the human brain, a guiding catheter would be chosen which would extend from the entry site at the femoral artery, up through the large arteries extending to the heart, around the heart through the aortic arch, and downstream through one of the arteries extending from the upper side of the aorta. A guidewire and neurovascular catheter such as that described in the Engelson patent are then placed through the guiding catheter. Once the distal end of the catheter is positioned at the site, often by locating its distal end through the use of radiopaque marker material and fluoroscopy, the catheter is cleared and/or flushed, for example, with an electrolyte solution.

Once the selected site has been reached, the vaso-occlusive device is extruded using a pusher-detachment mechanism as described herein and released in the desired position of the selected site.

Modifications of the procedure and catheters described above, and the methods of using them in keeping with the present disclosure will be apparent to those having skill in this mechanical and surgical art. These variations are intended to be within the scope of the claims that follow.

What is claimed is:

1. A catheter for use in delivering electrolytically detachable vaso-occlusive devices, the catheter having a distal region, sidewalls and a lumen therethrough, wherein the sidewalls of the distal region comprise one or more apertures therein.

2. The catheter according to claim 1, wherein the apertures comprise one or more slots or holes.

3. The catheter according to claim 1, wherein the distal region of the catheter comprises a porous membrane.

4. The catheter according to claim 2, wherein the distal region of the catheter further comprises a porous membrane.

5. The catheter according to claim 2, wherein the apertures comprise longitudinal slots.

6. The catheter according to claim 2, wherein the apertures comprise axially aligned or staggered holes.

7. A catheter according to claim 1, wherein the lumen of the catheter further comprises an electrolyte solution.

8. The catheter according to claim 7, wherein the electrolyte solution comprises saline or phosphate buffered saline.

9. A catheter for use in delivering electrolytically detachable vaso-occlusive devices, the catheter having a distal region, a lumen therethrough and means for maintaining current flow in the distal region of the catheter to obtain minimal resistive drop between the detachment site and the subject's blood.

10. A vaso-occlusive assembly comprising
a vaso-occlusive device comprising an electrolytically detachable junction; and
a catheter according to claim 1.

11. The assembly according to claim 10, wherein the apertures comprise one or more slots or holes.

12. The assembly according to claim 10, wherein the distal region of the catheter comprises a porous membrane.

13. The assembly according to claim 11, wherein the distal region of the catheter further comprises a porous membrane.

14. The assembly according to claim 10, wherein the apertures comprise longitudinal slots.

15. The assembly according to claim 10, wherein the apertures comprise axially aligned or staggered holes.

16. The assembly according to claim 10, wherein the lumen of the catheter further comprises an electrolyte solution.

17. A vaso-occlusive assembly comprising
a vaso-occlusive device comprising an electrolytically detachable junction; and
a catheter according to claim 9.

18. A method of at least partially occluding an aneurysm, the method comprising the step of introducing a vaso-occlusive device into the aneurysm using the vaso-occlusive assembly according to claim 10.

19. A method of at least partially occluding an aneurysm, the method comprising the step of introducing a vaso-occlusive device into the aneurysm using the vaso-occlusive assembly according to claim 17.
